(19) United States
(12) Patent Application Publication
(10) Pub. No.: US 2010/0211186 A1
(43) Pub. Date: Aug. 19, 2010

(54) ELECTROACTIVE POLYMER ACTUATION OF IMPLANTS

(75) Inventors: Craig M. Senders, El Macero, CA (US); Travis T. Tollefson, Sacramento, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 12/733,077

(22) PCT Filed: Aug. 8, 2008
(86) PCT No.: PCT/US08/09521

§ 371 Date: Apr. 27, 2010

Related U.S. Application Data
(60) Provisional application No. 60/964,293, filed on Aug. 9, 2007.

(51) Int. Cl. A61F 2/48

(52) U.S. Cl. 623/24

(57) ABSTRACT

This invention provides methods and devices for reconstructing muscular responses in patients with paralysis. Electroactive polymer (EAP) actuators power implants attached to tissues in the patients. When the actuators are energized, the implants move the tissues appropriately to provide improved body functions to patients experiencing a paralysis or paresis.

This application claims priority to and benefit of a prior U.S. Provisional Application No. 60/964,293, Electroactive Polymer Actuation of Implants, by Craig M. Senders, et al., filed Aug. 9, 2007. The full disclosure of the prior application is incorporated herein by reference.

### FIELD OF THE INVENTION

The invention is in the field of active reconstructive prosthetics. Electroactive polymer (EAP) actuators are employed to move implants thus moving tissues associated with the implants. Actuators are typically mounted to dense connective tissue of a patient, while the implant is attached to the tissue to be animated. The EAP actuators can be used to constrict lumens, open or close eyes, articulate joints, reproduce facial expressions, etc.

### BACKGROUND OF THE INVENTION

Most current prosthetics for reconstruction and reanimation of absent or paralyzed tissues are typically passive in function. The field of robotics has recently crossed over to provide externally worn prosthetics to replace lost limbs or to support the movement of disabled limbs. However, a need remains for internally implanted active prosthetics with more normal function and appearance.

The face is an area where natural appearing active prosthetics would be particularly welcome. The conventional treatment of permanent facial paralysis has included a variety of surgical procedures that endeavor to recreate the form and function of the facial musculature. For example, eyelid closure problems in facial paralysis not only can lead to corneal ulceration or blindness, but harms self-image and social acceptance. Conservative therapy consists of lubrication, moisture chambers, while surgical procedures have included tarsorrhaphy or gold weight implantation (Snyder M, Johnson P, Moore G et al., Early Versus Late Gold Weight Implantation for Rehabilitation of the Paralyzed Eyelid, Laryngoscope 111: 2109-2113, 2001). The main benefits of a gold weight placement are the effectiveness of corneal protection and ease of implantation. A major weaknesses of gold weight placement include creation of an blink that is unsynchronized with the normal eyelid due to a delay in gravity closure of the eyelid. Moreover, incomplete medial eyelid closure (medial gaposis) can occur with a gold weight that is placed too laterally on the tarsal plate. In other methods of active blinking reconstruction have used surgical transposition of a small segment of temporalis muscle fascia (Gillies S H and Millard D R. The Principles and Art of Plastic Surgery. London: 1957; Frey M, Giovanoli P, Tzou C J, et al. Dynamic Reconstruction of Eye Closure by Muscle Transposition or Functional Muscle Transposition in Facial Palsy; Plast Reconst Surg 114: 865-873, 2004). Free tissue transfer from the serratus or gracilis muscle at the time of lower facial sling procedures has also been reported (Harii K, Ohmori K, Murakami F., Free, Gracilis Muscle Transplantation with Micro Neurovascular Anastomoses for the Treatment of the Facial Paralysis, Plast Reconstr Surg 2:133-143, 1976). However, these techniques have significant issues with reperfusion and innervation.

The field of robotics has contributed increasingly efficient devices capable of walking, jumping, and running similar to humans. Technologies such as electromagnetics, mechanochemical polymers, electrochemomechanical polymers (conducting polymers), magnetostrictive materials, piezoelectric, hydraulics and pneumatics, thermal expansion, and fuel burning engines have been used for robotic movement. The performance of these substances is different from that of biologic muscle in terms of force, speed, and strain.

In the 1990's, the Stanford Research Institute (SRI) in Menlo Park, Calif. developed electroactive polymer artificial muscle (EPAM) which is now licensed exclusively to Artificial Muscle, Inc., an SRI spin-off company, (Kornbluh R, Pelrine R, Eckerle J, Joseph, J., Electrostrictive Polymer Artificial Muscle Actuators, Robotics and Automation Proceedings 3: 2147-2154, 1998). EPAM is a lightweight, highly efficient alternative to small motors, capable of generating muscle-like motion (e.g., actuation, translation) in response to a low currents or voltages. Potential uses being investigated for EPAMs include mobile robots for use in space, movement of liquids in a manner similar to hydraulics systems, and as diaphragms for speakers. However, problems with biocompatibility, mechanical configuration and provision of power have limited EPAM utility an internally implanted active prosthetics.

In view of the above, a need exists for internally mounted biocompatible active prosthetics. It would be desirable to have concealed prosthetics to activate facial expressions. The present invention provides these and other features that will be apparent upon review of the following.

### SUMMARY OF THE INVENTION

The present invention includes implantable active prosthetic devices and methods for animation of paralyzed tissues. The devices can include, e.g., electroactive polymer actuators that move implants associated with tissues of the patient. The prosthetic systems can include power supplies to energize the devices and sensors to trigger movement of the implants. The methods include fabrication of the devices, surgical implantation of the devices and use of the devices to provide improved function of dysfunctional tissues.

It is a useful and novel aspect of the inventions that the actuator of the devices is not required to be in direct contact with the tissue for the implant to move the tissue. The actuator and implant can be specialized, e.g., in motion generation and motion transduction functions.

The electroactive prosthetic devices of the invention can include, e.g., an electroactive polymer actuator and a biocompatible implant functionally attached to the actuator so that application of a voltage to the actuator mechanically translates the implant. In certain embodiments, the device includes an electroactive polymer actuator, a biocompatible cord implant functionally attached to the actuator, and a source of electrical voltage in controllable electrical contact with the actuator. The biocompatible cord can be configured for attachment to tissues of a living animal and to move the tissue on application of the voltage to the actuator.

The devices can include a sensor configured to energize the actuator. Exemplary sensors include, e.g., optical sensors, optical blink sensors, myoelectric sensors, position sensors, and/or the like.

The power source can be in electrical contact with the actuator can be, e.g., a battery, a fuel cell, an induction coil, a capacitor and/or the like.

The device can be configured for attachment to a tissue or organ to provide motion of the tissue or organ. For example, the tissue or organ can include an eyelid, facial skin, a facial muscle attachment point, an eyebrow, a bone, a diaphragm, a finger, a hand and/or the like.

The actuator can include a covering, such as a case, sheath, or capsule. Typically, the cover is waterproof and preferably biocompatible. In many cases, the cover has an outer surface composed of, e.g., polyvinyl chloride, silicone, polyurethane, ligament, a connective tissue, polytetrafluoroethylene, polycarbonate, polyester, polyethylene, a hydrogel, titanium, stainless steel and/or the like.

The electroactive polymer acts to contract or expand in response to a change in applied voltage. The electroactive polymer (EAP) can be an electronic electroactive polymer or an ionic electroactive polymer. For example, the EAP can be an electro-statically stricted polymer, an electronic electroactive polymer, an ionic electroactive polymer, polyaniline, polypyrrole, polyacetylene, and/or the like.

The implants of the device can be in the form of a sling, a cord, a membrane, a lever, a detent, a pulley or a construct mimicking the shape of a tissue to be replaced. The implants are typically fabricated from a biocompatible material, such as, e.g., a ligament, a connective tissue, a suture, polyvinyl chloride, silicone, polyurethane, polytetrafluoroethylene, polycarbonate, polyester, polyethylene, a hydrogel, titanium, stainless steel, and/or the like.

The methods of using an electroactive prosthetic devices include, e.g., providing an electroactive polymer actuator functionally attached to a biocompatible implant, attaching the implant to one or more transposition points of an internal tissue of a living animal (e.g., a human), mounting the actuator to an anchoring location on the animal, and applying a voltage to the actuator, thereby mechanically translating the implant and the tissue. In one embodiment, the method of using an electroactive prosthetic device includes providing an electroactive polymer actuator attached to one or more biocompatible implant slings, attaching the one or more slings to a medial orbit of an eye socket, running the one or more slings along one or more transposition points in tissue of one or more eyelids, slidably mounting the one or more slings at a lateral orbit of the eye socket, mounting the actuator at an anchoring location outside the eye socket, and applying a voltage to the actuator. The pulling force of the actuator can pull on the one or more of the slings to urge closure of the one or more eyelids.

To provide for eyelid opening, the method can further comprise providing a second electroactive polymer actuator functionally attached to a biocompatible cord and mounted at an anchoring location above the orbit of the eye socket. The cord can be attached to a transposition point of the upper eyelid so that application of a voltage to the actuator will pull up on the cord to open the upper eyelid.

The methods can include internally mounting a power supply to provide voltage. As stated above, typical power supplies can be, e.g., a battery, a fuel cell, and an induction coil. In many embodiments, application of voltage to the actuator is in response to a signal from a sensor.

In the methods, the actuator can have a mounting case with a biocompatible outer surface. The electroactive polymer can be an electro-statically stricted polymer, an electronic electroactive polymer, an ionic electroactive polymer, polyaniline, polypyrrole, polyacetylene or the like. The sling can be fabricated from a ligament, a connective tissue, a suture, polyvinyl chloride, silicone, polyester, polytetrafluoroethylene, polyethylene and/or the like.

The implant can be attached to tissue of a patient at one or more transposition points where force and movement from the actuator can influence the tissue. Typical transposition points for association with implants are, e.g., connective tissue of an organ, skin connective tissue, a bone surface, a muscle attachment point, a tendon, and/or the like. In an eye closure prosthetic, it is preferred the tissue for sling attachment is the eyelid tarsal plate. In a preferred embodiment, the one or more slings run through the top eyelid and through the bottom eyelid. A preferred approach at the lateral eye orbit for the sling is from a slidable mount just adjacent (e.g., adjoining or within 1 mm, 3 mm, 5 mm, or 10 mm) above Whitnall's tubercle. It is preferred in the eye closure device that the actuator anchoring location be on, e.g., the temporal bone, zygomatic bone, or sphenoid bone.

### DEFINITIONS

Unless otherwise defined herein or below in the remainder of the specification, all technical and scientific terms used herein have meanings commonly understood by those of ordinary skill in the art to which the present invention belongs.

Before describing the present invention in detail, it is to be understood that this invention is not limited to particular devices or methods, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a component” can include a combination of two or more components; reference to “a polymer” can include mixtures of polymers, and the like.

Although many methods and materials similar, modified, or equivalent to those described herein can be used in the practice of the present invention without undue experimentation, the preferred materials and methods are described herein. In describing and claiming the present invention, the following terminology will be used in accordance with the definitions set out below.

The term “electroactive polymer”, as used herein, refers to polymers that change in size or shape in response to application of an electrical voltage or electric current. For example, electroactive polymers of the invention can optionally be piezoelectric polymers, ionic electroactive polymers (e.g., certain conductive polymers, electrorheological fluids, ionic polymer gels, or ionic polymer metallic composites), or electronic electroactive polymers (e.g., certain sandwiched dielectric EAPs, electrostrictive graft elastomers, electrostrictive paper, electro-viscoelastic elastomers or ferroelectric polymers).

An “implant”, with regard to devices of the invention is a component of the device that is functionally attached to the EAP for movement by the EAP, but does not comprise the EAP. In use, the implant functions to convey motion from the EAP to a tissue intended to be moved. The cover around the EAP in an actuator is not considered to be the implant of the inventive devices. An implant is typically intended for use implanted in the tissues of a patient with all or part of the implant internal to the integument of the patient.

An implant can be considered “biocompatible” if, on implantation, the material does not cause injury, toxic or immunologic reaction to living tissue. Preferred biocompatible materials are not dissolved or actively resorbed in the body.

A “sling”, as used herein, refers to a cord in a relaxed condition that does not define a line. For example, a sling can be a cord hanging in a parabolic loop. However, when tension is provided along the sling, it can be forced to approach or achieve linear geometry. During such motion, e.g., tissue associated with the sling can move accordingly.

An “actuator” is a component or device that provides movement to an associated object or article. For example, an electroactive polymer actuator is an actuator with force and movement generated by an EAP, which can cause an associated implant to move when the actuator is energized and/or deenergized.

“Translation”, as used herein, refers to physical movement from one position or location to another.

A “mounted” component of a mechanical device is mounted directly to the indicated mounting location, unless noted otherwise.

The term “adjacent”, as used herein, means adjoining or relatively near in the scale of the subject objects. For example, a cord run adjacent to an anatomical landmark is at the outer boundary of the landmark or within, e.g., 5 mm, of the landmark.

An “anchoring location” is a mounting location in a tissue for implanted actuator mounting hardware. For example, the anchoring location can be the site on a bone where a hard cased actuator is cemented. Optionally, the anchoring location can be the position in a connective tissue of a patient where the actuator is attached with sutures, pins, screws, etc. An “internal” anchoring location is under the skin of the animal.

A “transposition point” is a tissue point of attachment or contact with an implant that allows the tissue to be moved by translation of the implant. The implant can be fixedly attached to the tissue at the transposition point, slidably attached, or passively associated to transfer movement of the implant to the tissue at the transposition point.

Directional anatomical terminology are to be interpreted as for the indicated animal in the standing position and according to standard directions and planes in the field of anatomy.

### BRIEF DESCRIPTION OF THE DRAWINGS

FIG. 1 is a schematic diagram of an electronic electroactive polymer.

FIG. 2 is a schematic diagram of an electroactive prosthesis wherein the implant is a linear cord.

FIG. 3 is a schematic diagram of an electroactive prosthesis wherein the implant is a cord configured as a sling.

FIG. 4 is a schematic diagram of a prosthetic system of the invention for closure of eyelids.

### DETAILED DESCRIPTION

The devices and methods of the invention allow one to controllably move a tissue, organ or suborgan, e.g., to replicate functions lost in a disabled patient. The devices include, e.g., an electroactive polymer actuator functionally interacting with a biocompatible implant and powered by an electrical power supply. The implant can be fixedly mounted to a tissue of a patient to transfer the actuator motion or force to the tissue. Methods of using the electroactive polymer prosthetic devices can include, e.g., fabricating an actuator that functions to move an implant, surgically mounting the actuator to a secure and properly aligned location in the body of the patient and attaching the implant to a tissue to be moved by activation of the actuator. A voltage can be supplied to the actuator in a fashion controllable, e.g., by a manual switch or according to signals sent from appropriate sensors.

### Electroactive Polymer Prosthetic Devices

In general, electroactive polymer (EAP) prosthetic devices of the invention include a biocompatible implant that can be mechanically translated by an EAP actuator to appropriately move tissues associated with the implant. The devices can include a power supply, power control circuitry, sensors, and/or mounting hardware. In use, the devices can be implanted under the skin of a patient typically with the actuator fixedly mounted to a bone near a tissue to be moved, and the implant (e.g., cord, lever or membrane) fixedly or slidably attached to the tissue.

### Electroactive Polymer Actuators

Electroactive polymer actuators of the invention are typically electroactive polymers accessorized function as an actuator when implanted into an animal's body. The EAPs can have a cover, e.g., casing, to provide biocompatibility, electrical isolation, ion impermeability, power access, secure mounting points, and/or attachment points for implants to be moved.

The EAPs of the actuators can be of any type appropriate to the particular application. Typical EAPs are change size in at least one dimension in response to either electronic stimulation or changes to the surrounding ionic environment. For example, electroactive polymers of the invention can optionally be piezoelectric polymers, ionic electroactive polymers (e.g., certain conductive polymers, electrorheological fluids, ionic polymer gels, or ionic polymer metallic composites), or electronic electroactive polymers (e.g., certain dielectric EAPs, electrostrictive graft elastomers, electrostrictive paper, electro-viscoelastic elastomers or ferroelectric polymers).

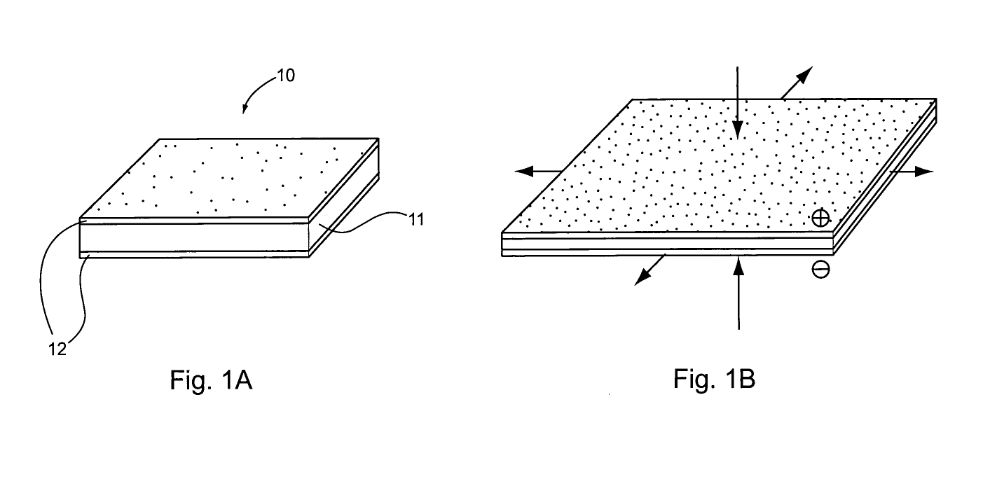

The basic mechanism of motion generation (actuation) provided by a typical dielectric EAP depends on a three-layer material structure, as shown in FIG. 1. A thin film (e.g., 20 to 60 microns) of a dielectric elastomer, such as silicone or acrylic, is layered on each side with a conductive electrode layer made of carbon particles suspended in a soft polymer matrix. The electronic EAP 10 comprises an elastomeric dielectric polymer film 11 sandwiched between compliant electrodes 12, as shown in FIG. 1. When a DC voltage is applied to the electrodes, as shown in FIG. 1B, the attraction between the electrodes squeezes the elastomeric polymer causing it to expand in a plane perpendicular to the force. The outward force of such an expansion, or the retractive recoil on elimination of the voltage, can be used to move an attached implant. The voltages required for such a device can be relatively high, but current requirements can be quite low. When the voltage differential across the electrodes is neutralized, the elastomer polymer can contract back to the original shape with substantial force, so that an attached implant can be pulled back to an original position. The advantages of these “capacitor” style EAPs can be low current usage, and the ability to provide ample forces across useful distances. A disadvantage in biological systems can be the need to electrically insulate the components against a relatively high working voltage.

The qualities of dielectric EAPs which make them good candidates for an biologically compatible artificial muscle include: 1) thin size, 2) ability to expand by as much as 400 percent or more when voltage is applied, 3) an ability to provide a strong mechanical force, 4) minimal heat generation, and 5) minimal current needed to maintain an activated state (expanded). In general, a linear relationship of voltage to device expansion is noted until a threshold of voltage is exceeded. At this point, increasing voltage result in less expansion as current flows dielectric silicone.

Ionic EAPs, such as ionic polymer gels, ionomeric polymer-metal composites, conductive polymers and certain carbon nanotubes, work on the basis of electrochemistry. Voltages required to affect ion interactions or electroosmotic flows can be relatively low, but the currents can be significant. Exposure to excessive voltages can cause problems with electrolytic production of heat and gasses. Moreover, in many cases, the ionic environment around the ionic polymers must be maintained within a certain range for the material to operate properly.

EAPs for use as implanted actuators are typically sealed in a flexible cover or hard case. For example an electronic EAP can be covered in a flexible plastic insulator, at least at the electrodes and power supply wire contacts. Tough contact areas can be provided for attachment to implants and mounting hardware. Ionic EAPs can be covered in a flexible capsule, which is impermeable to movement of ions into or out of the capsule interior. Alternately, the EAPs can be enclosed in a hard capsule or case that provides electrical insulation and/or isolation of ions, as required. When the EAP cover is a hard case, a sealed port is typically required to allow force or motion of the EAP to be received by the implant. For example, the EAP can connect to the implant through a port sealed with a flexible membrane or an o-ring.

The cover or case can include a means to fixedly mount the EAP to a structural support in the body of a patient. For example, the case can include a hole or grommet to receive a surgical screw or pin for attachment to bone. Optionally, the cover or case can have, e.g., a biocompatible membrane or fabric allowing the case to be attached with sutures to connective tissues, such as tendons, ligaments, cartilage, aponeuroses, skin attachment points of platysma, and the like.

It is preferred the outer surface of the actuator be covered in a biocompatible, non-immunogenic material. For example, the EAP actuator can be covered with polyvinyl chloride, silicone, polyurethane, polytetrafluoroethylene, polycarbonate, polyethylene, titanium, stainless steel and/or the like.

### Biocompatible Implants

The implants of the present devices function to transfer the force or motion of the EAPs to do work on tissues, as desired. The implants can be mechanical structures of suitable geometry and mechanical strength to perform a particular tissue translating task at hand. Depending, e.g., on the force and “throw” of a particular EAP employed, the implant can be configured to provide the desired mechanical advantage and/or force vectors for the job. The implants are structures that can do the specialized mechanical work of the devices while allowing the EAPs to function in the compatible internal environment of the actuator.

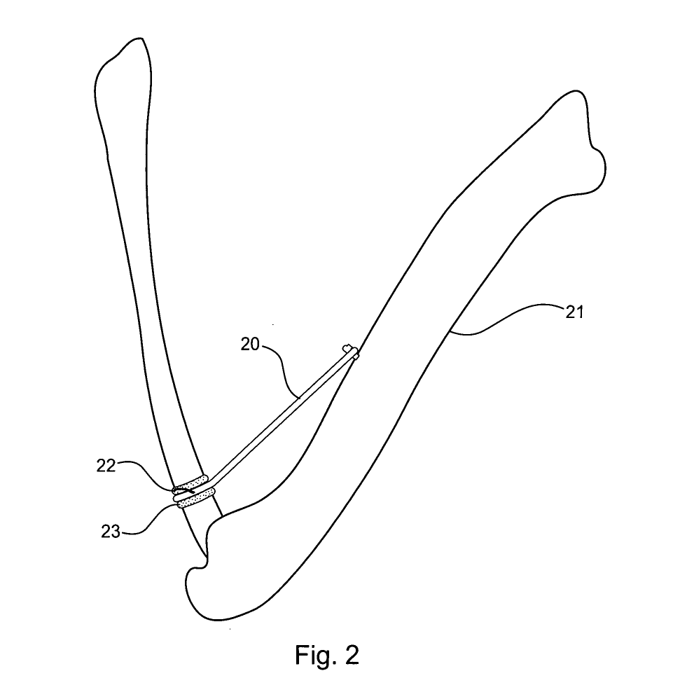

Implants can be unitary components or include multiple interacting parts. In a typical embodiment, an implant of the invention is simply a cord 20 intended to pull on a tissue 21 in response to a constrictive movement of the EAP 22 in an actuator 23, as shown in FIG. 2. The cord can be, e.g., a line, string, filament, wire, etc; single stranded or multi-stranded; flexible or rigid. The cord can be mounted between the EAP and tissue to provide a direct linear pull, e.g., with the tissue attached at one end of the cord and the EAP at the other end of the cord implant.

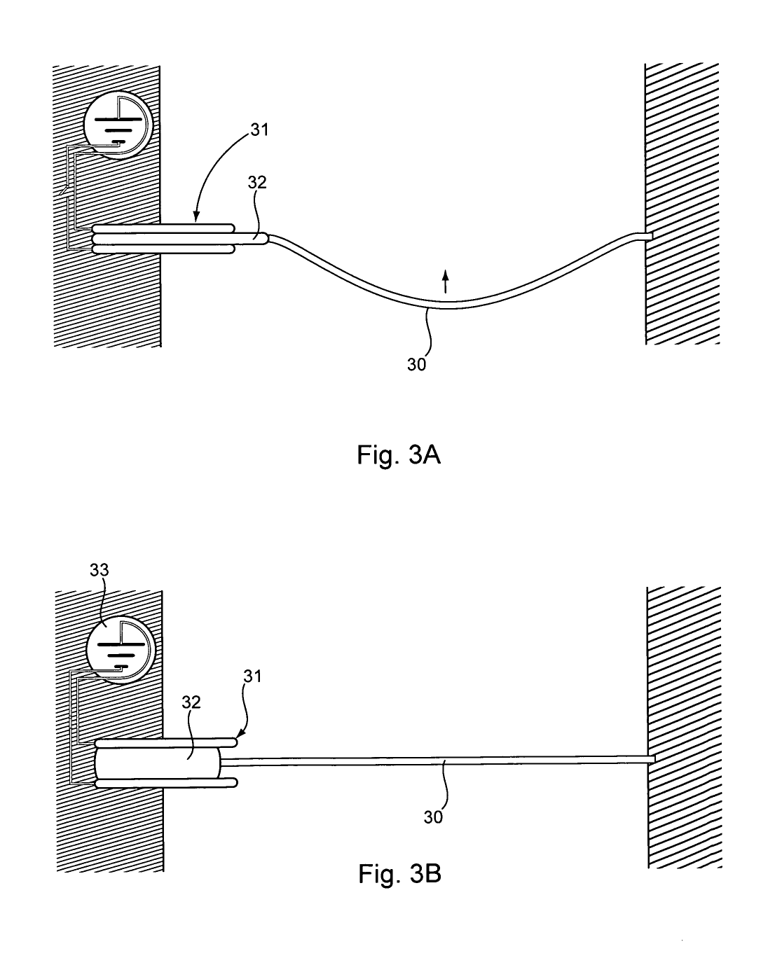

Optionally, the cord implant can be mounted as a sling 30, e.g., loosely mounted between the actuator 31 and a fixed mount point (e.g., on a solid tissue intended to remain relatively motionless) with excess cord displaced laterally, as shown in FIG. 3A, while the actuator EAP 32 is in an extended position. When a voltage is supplied (or eliminated, e.g., in the case of certain dielectric EAPs) from voltage source 33, actuator 31 can be established in a retracted position. As shown in FIG. 3B, the sling 30 can be pulled taught (or at least toward a more linear geometry) so that displaced portions of the sling are moved, e.g., toward a center line between the actuator and the fixed mount point. Tissue can be fixedly or slidably attached to the sling to be pulled laterally when the actuator is in a retracted position.

Alternately, the implant can be provided in a constrictive loop or noose configuration. Such configurations can aid in constrictions of body system lumens, such as found in the vasculature, alimentary tract, urinary systems, and the like. In constrictive embodiments, the actuator can be mounted on or near the body conduit to be constricted. The implant can be wrapped around the conduit. When the actuator is in the retracted condition, the implant can act to constrict the body conduit and reduce or eliminate the internal lumen cross section. When the actuator is in the expanded condition, the implant cord can be relaxed, allowing expansion (e.g., dilation) of the lumen cross section.

In other embodiments, the implant can have one or more planar or membranous components. For example, the implant can be a concave shaped membrane that can be pulled peripherally by EAP actuators to urge the membrane into a more flattened topography. Such an implant could act as a diaphragm or as part of a fluid pumping mechanism.

Depending on the force and throw of the EAP and actuator assembly, it may be desirable to configure the implant to provide a mechanical advantage. For example, where the actuator is providing a large force with a short throw (over a short distance), and where it is desired to move a tissue over a larger distance with a lesser force, the implant can be designed as a lever with a pivot point so that the EAP pulls on a short arm of the lever and the tissue is attached at the end, or along, the long arm of the lever. For example, the implant could be a rigid lever pivoting from the hard case of the actuator, with the EAP attached close to the pivot point. The implant arm can extend out to a tissue to move the tissue a multiple of the distance from the pivot point to the EAP attachment point, but with an inverse fraction of the force, as is known in the art.

In preferred embodiments, the implants are one or more biocompatible materials that do not degrade in situ and do not elicit inflammation at the implantation site. The biocompatible implant material can be of a natural material, such as connective tissue from the same patient into whom the implant will be implanted. Optionally, the implant can be formed from allograft connective tissue, a ligament, suture material, polyvinyl chloride, silicone, polyurethane, polytetrafluoroethylene, polycarbonate, polyester, polyethylene, a hydrogel, titanium, stainless steel and/or the like.

The implant can be associated with tissue so as to transfer motion of the implant to the tissue. In most cases, this entails a fixed or slidable attachment of the implant to the tissue. Any appropriate means of attachment can be used between the implant and the tissue, e.g., a suture, pin, screw, staple, a biologic glue, a cement, and/or the like. In some cases, the implant is also mounted directly to a tissue that is not intended to be moved by the implant. For example, where the tissue is intended to be moved by lateral movement of a sling, the end of the sling can be fixedly mounted to a relatively stationary bone while fixed to the tissue to be moved, or slidably intertwined in the tissue to be moved.

### Power Supplies

Electroactive polymer prosthetic devices have actuators that require a voltage source to function. In many cases, the devices require significant amounts of current to function over extensive periods of time. Typically, the devices are powered by a battery of some type. The power supply can be housed in the case of the actuator, or mounted at a location separate from the actuator.

In most cases, it is desirable to have the power supply for the electroactive device implanted under the skin of the patient. In cases where the current requirements are small (e.g., short term prosthetics, prosthetics with small power demands), a single use battery may be adequate. The battery can be of any appropriate type, such as, e.g., silver chloride, alkaline, NiCad, lithium, fuel cells, etc. It can be useful to have rechargeable batteries, e.g., rechargeable through external contacts or through transdermal electromagnetic induction to an implanted coil. The devices can be powered by a generator implanted in contact with moving body parts to provide the required power or battery recharge.

Battery power can be conserved by turning off the device while it is not in use. A switch can be provided in the device circuitry to shut off the device, e.g., while the patient is sleeping. A manual switch can be provided internally or externally. Optionally, a magnetic or radio frequency switch can be provided, e.g., to toggle on and off in response to appropriate electromagnetic signals.

### Sensors

In many cases, it is useful to control energization of the actuators based on detection of a condition. For example, actuators can be triggered to translate an associated tissue in response to a signal from a sensor. The sensor can be, e.g., a timer, a position sensor, an optical sensor, an electrode, a contact sensor, and/or the like.

For example, an optical sensor can detect a projectile near the eye and signal a blink. Where the prosthetic device is intended to provide a blink function to a paralytic eyelid, a timer can provide a signal for periodic blink actuation.

Sensor and signal systems can be used to synchronize (or, in some cases, intentionally asynchronize) actuators with patient movements. For example, where one eye is not paralyzed, an optical sensor (e.g., mounted to a glasses frame) can detect blinking of the functional eye and synchronize blinking of the dysfunctional eye. Optionally, a myoelectric sensor monitoring, e.g., orbicularis oculi muscle (blinking) of the functional eyelid can be used to signal synchronized blinking of the dysfunctional eyelid.

In other embodiments, a contact sensor can be used to appropriately signal actuation. For example, a contact sensor in a glove can signal actuation of a device to open or close a hand.

### Exemplary Embodiment of EAP Prosthetic Device

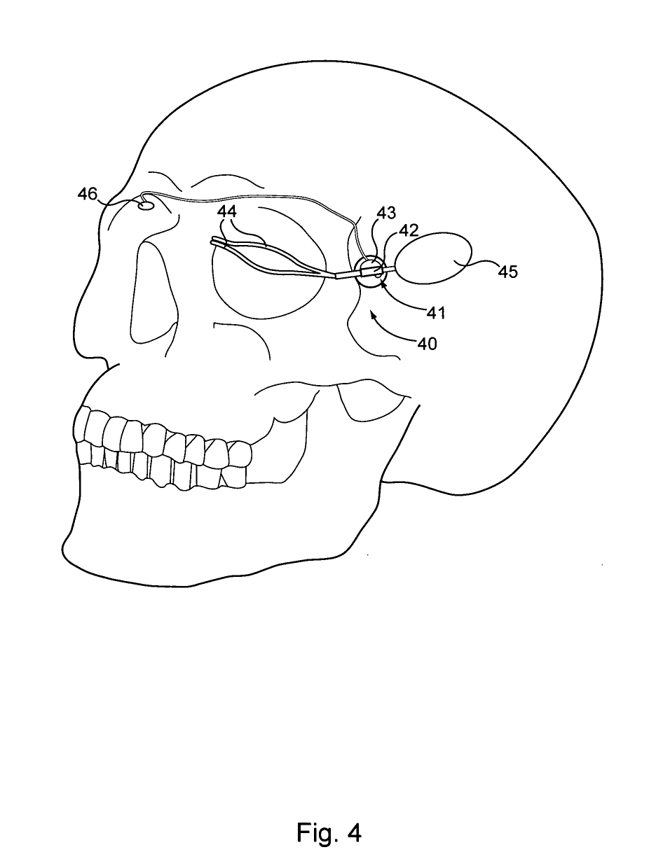

A typical embodiment of the inventive devices is a system for providing eye closure to paralytic eyelids. As shown in FIG. 4, an EAP device 40 includes an actuator 41 having an electroactive polymer 42 in a biocompatible cover 43. The electroactive polymer is attached to a pair of cord implants 44 and energized by a power supply 45. A myoelectric sensor 46 is in electrical contact with the actuator to provide an actuation signal.

In use, the device is surgically implanted into a patient in need. Through one or more incisions, the pair of sling cord implants are anchored to the medial orbit of the eye and run along tissue of the eyelids and around the zygomatic bone at the lateral orbit of the eye. The actuator is mounted at an anchor location on the sphenoid bone. The power supply is placed over the auricularis muscles in electrical contact with the actuator. The myoelectric sensor is placed in contact with the orbicularis muscle and a signal line is run through the scalp to a signal processing circuit housed in the actuator. All incisions are closed and the patient is allowed to heal.

The implanted system functions to close the paralytic eyelid whenever the functional eyelid is closed. For example, when the functional eye of the patient blinks, the muscle activity is picked up by the sensor, which sends a signal to the signal processing circuit. The circuit appropriately configures electrical contacts to initiate a contraction of the EAP in the actuator, thereby pulling on the sling cords and closing the eyelids over the eye.

### Methods of Using Electroactive Polymer Prosthetic Devices

Electroactive polymer prosthetic devices of the invention can be employed by attaching the actuator and implant to tissues of a patient, in a manner appropriate to the regain a disabled function. Typically, in a surgical procedure, the actuator and implant are attached to tissues at two different locations. In many embodiments, the actuator is fixedly attached to a relatively stationary solid tissue structure and the implant is attached to a tissue to be moved by the actuator. In many embodiments, the device can replicate the function of a particular non-functional muscle with actuator and implant attachment points at or adjacent to the attachment points of the muscle. In other cases practical considerations necessitate novel arrangements to accomplish the desired animations.

The methods generally include manufacture of the EAP device with an actuator attached to an implant, creating an incision near the device implantation site, inserting the device through the incision, running the implant through tissues to the transposition points and mounting the actuator at an anchoring location.

In one embodiment, the actuator is fixedly mounted to bone, or other dense connective tissue, and the implant is a cord attached to move a softer connective tissue or sub organ. For example, an upper eyelid can be opened by mounting an actuator at an anchoring location above the eyebrow to pull on an implant attached to the tarsal plate of the eyelid. In another embodiment of the invention, aspects of a smile can be reproduced, e.g., by mounting an EAP device of the invention between the zygomatic arch and connective tissue at the corner of a patient's mouth.

In some embodiments, implants do not run directly from the actuator to the tissue for to be moved. For example, a cord type implant can be slidably run around a “corner” between the actuator and the tissue. The point of sliding contact can be at a bone, a hole through a bone, or sheath of a connective tissue. Optionally, the point of sliding contact can be a surgically implanted material, such as a TEFLON or stainless steel sliding surface.

### EXAMPLES

The following examples are offered to illustrate, but not to limit the claimed invention.

Some potential uses for artificial muscle in humans include breathing with an artificial diaphragm, movement of fingers and hands, and facial reanimation. Of these concepts, we first looked at eyelid closure in the setting of facial paralysis. Such a closure was expected to require the relatively small forces and require relatively simple surgery for mounting of the implant.

### Example 1

### Routing of Slings for Animation of Eye Blinking

The authors' hypothesis is that a reproducible, long-lasting eyelid blink can be restored in patients with facial paralysis using EPAM. This preliminary study was conducted as a proof of concept to determine if a novel eyelid sling can be implanted to achieve closure of the eyelids of a cadaver.

Electroactive polymer artificial muscle (EPAM) is an emerging technology that we believe has the potential to be used in rehabilitating facial movement in patients with paralysis. Developed by the Stanford Research Institute (SRI, Menlo Park, Calif.), these electroactive polymers can act like human muscles by expanding and contracting based on variable voltage input levels. We examined the reanimation of smaller muscle groups, such as those responsible for eyelid closure or facial expression. Indications for such rehabilitation may include acquired facial paralysis after oncologic resection or traumatic injury. Other patients that may benefit from artificial muscle technology include those with congenital facial nerve disorders such as Moebius syndrome.

EPAMs can act like human muscles by expanding and contracting based on variable voltage input levels. We seek to establish a reproducible eyelid blink with artificial muscle. The aim of this proof of concept study was to determine if eyelid closure can be created with a novel eyelid sling model.

A cadaver model was developed to test the concept of an eyelid sling, which will transduce the EPAM contraction to the eyelids. Fresh cadaver heads from the UC Davis Body Donor Program were used in accordance with UC Davis Policies and Procedures. One eye of each cadaver was utilized for the proof of concept experiment. The opposite eye was intended for subsequent study of vector and force requirements and prototype design analysis.

Using four cadaver heads, an extended upper and lower blepharoplasty incision was used to secure an upper and lower, expanded polytetraflouroethylene implant to the medial orbital wall and tarsal plates. The slings were passed through a hole drilled in the lateral orbital wall or around a titanium screw. Lateral pull on the sling created eyelid closure and the necessary distance of pull was measured.

Dissection technique was as follows. The initial incision was a standard upper blepharoplasty incision at the supraciliary crease (about 9-10 mm above the lash line). The skin and orbicularis oculi muscle were carefully divided using a 15 blade with particular attention to preservation of the levator aponeurosis. The superior border of the tarsal plate in the upper eyelid was exposed (similar to that used in gold weight placement). To simulate the normal eyelid opening of the levator muscle, a 5-O-nylon suture was secured through the midline levator aponeurosis attachment to the tarsal plate and passed under the skin to exit in the upper eyebrow region.

A medial ePTFE insertion site was selected. Dissection superficial to the tarsal plate (similar to central fat pocket dissection in blepharoplasty) was carried medially to the junction of the frontal process of the maxilla and frontal bone. The lacrimal puncta was canulated with a lacrimal probe until the medial canthal dissection was completed. The attachments of the medial canthal (palpebral) ligament to the periosteum of the medial orbital wall was isolated and maintained. Dissection in a subperiosteal plane with a Freer elevator was performed just anterior to the anterior lacrimal crest and inferior to the junction of the frontal process of the maxilla and frontal bone. A 1.8 mm×60 mm expanded polytetraflouroethylene (ePTFE, Advanta implants, Atrium Medical Corporation, Hudson, N.H.) facial implant was secured under a 1.2 mm (2 hole) titanium plate with two titanium screws (1.2 mm diameter, 3 mm long, Stryker Leibinger Inc, Kalamazoo, Mich.). The screws were secured anterior to the medial canthal attachments. Three 6-0 silk sutures were used to attach the ePFE sling to the perichondrium of the tarsal plate (similar to securing a gold weight). The skin was approximated. The lower lid (subciliary) approach was used for the lower eyelid sling to be placed.

In this eyelid sling concept, a laterally directed force on the sling pulls the sling from an arched shape to a linear shape, thereby closing the eyelids. The point at which the sling passes over the lateral orbital rim was tested with two techniques. First, a hole was drilled in the lateral orbital bone proximal to Whitnall's tubercle (as described by Alex et al for transorbital facial suspension). The slings for upper and lower eyelids were passed through the hole and tension applied until the eyelids were closed. The distance of sling displacement through the lateral orbital drill hole was measured. In a second technique, a titanium screw was placed into the lateral orbital rim as a positioning guide for the slings. The slings were passed around the screw. The required distance of pull was measured.

The approach was successful at positioning the eEPTE sling in the upper and lower eyelids to create a sphincteric-like closure of the eyelids when the sling was pulled laterally. The distance of pull required to create complete eyelid closure was 3 mm when both slings were activated, while 6 mm of sling displacement when pulling only the upper sling. The positioning of the lateral orbital wall drill hole was noted to be above Whitnall's tubercle (attachment of the lateral canthal tendon) and approximately 1 cm inferior to the frontozygomatic suture line. In one dissection, the drill hole was positioned higher and did not allow for complete eyelid closure laterally due to the inadequate vector. Correction with a drill hole placed just above Whitnall's tubercle eliminated lagopthalmosis.

This preliminary study established a possible method of converting the energy created by artificial muscle (EPAM) into a reproducible eyelid closure. We have found the required pull distance and required force to be well within the capabilities of available EPAMs.

An electroactive polymer will be able to be implanted under the skin with a self-contained energy source as a biologically active, artificial muscle. For comparison, a 1.2 gram gold weight implant that has been placed in an upper eyelid facilitates eyelid closure with the force of gravity. The EPAM device will need to create a similar force to close the eyelids. Because one Newton of force is equal to about 102 grams on the surface of the Earth, significantly less than a Newton of force will likely be needed to close the eyelids. This is not a difficult force to obtain using electroactive polymer actuators.

It is understood that the examples and embodiments described herein are for illustrative purposes only and that various modifications or changes in light thereof will be suggested to persons skilled in the art and are to be included within the spirit and purview of this application and scope of the appended claims.

While the foregoing invention has been described in some detail for purposes of clarity and understanding, it will be clear to one skilled in the art from a reading of this disclosure that various changes in form and detail can be made without departing from the true scope of the invention. For example, many of the techniques and apparatus described above can be used in various combinations.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. An electroactive prosthetic device comprising:
an electroactive polymer actuator;
a cord implant functionally attached to the actuator; and,
a source of electrical voltage in controllable electrical contact with the actuator;
wherein the biocompatible cord is configured for attachment to tissues of a living animal and to move the tissue on application of the voltage to the actuator.

2. The device of claim 1, wherein the activator is not in direct contact with the tissues when the implant is attached to the tissues.

3. The device of claim 1, further comprising a sensor configured to energize the actuator.

4. The device of claim 3, wherein the sensor is selected from the group consisting of: an optical sensor, an optical blink sensor, a myoelectric sensor, and a position sensor.

5. The device of claim 1, further comprising a voltage source in electrical contact with the actuator, wherein the voltage source is selected from the group consisting of: a battery, a fuel cell, and an induction coil.

6. A method of using an electroactive prosthetic device comprising:
providing an electroactive polymer actuator attached to one or more implant slings;
attaching the one or more slings to a medial orbit of an eye socket;
running the one or more slings along one or more transposition points in tissue of one or more eyelids;
slidably mounting the one or more slings at a lateral orbit of the eye socket;
mounting the actuator at an anchoring location outside the eye socket; and,
applying a voltage to the actuator thereby pulling on one or more of the slings, thus urging closure of the one or more eyelids.

7. The method of claim 6, wherein the actuator comprises a mounting shell with a biocompatible outer surface.

8. The method of claim 6, wherein the electroactive polymer is selected form the group consisting of: an electro-statically stricted polymer, an electronic electroactive polymer, an ionic electroactive polymer, polyaniline, polypyrrole and polyacetylene.

9. The method of claim 6, wherein the sling comprises a material selected from the group consisting of: a ligament, a connective tissue, a suture, polyvinyl chloride, silicone, polyester, polytetrafluoroethylene, and polyethylene.

10. The method of claim 6, wherein the tissue of an eyelid comprises a tarsal plate.

11. The method of claim 6, wherein the one or more slings run through a top eyelid and through a bottom eyelid.

12. The method of claim 6, wherein the sling slidable mount is adjacent above Whitnall's tubercle.

13. The method of claim 6, wherein the actuator anchoring location comprises a bone selected from the group consisting of: a temporal bone, zygomatic bone, or sphenoid bone.

14. The method of claim 6, further comprising internally mounting a power supply to provide the voltage.

15. The method of claim 6, wherein the voltage is provided from an electrical source selected from the group consisting of: a battery, a fuel cell, and an induction coil.

16. The method of claim 6, further comprising:
providing a second electroactive polymer actuator functionally attached to a cord implant and mounted at an anchoring location above the orbit of the eye socket;
attaching the cord to a transposition point of an upper eyelid of the one or more eyelids;
applying a voltage to the actuator, thereby pulling up on the cord to open the upper eyelid.

17. An electroactive prosthetic device comprising:
an electroactive polymer actuator; and,
a implant functionally attached to the actuator;
whereby application of a voltage to the actuator mechanically translates the implant.

18. The device of claim 17, wherein the device is configured for attachment of the implant to a tissue or organ to provide motion of the tissue or organ.

19. The device of claim 18, wherein the actuator does not have to be in direct contact with the tissue or organ to provide the motion.

20. The device of claim 18, wherein the tissue or organ is selected from the group consisting of: an eyelid, facial skin, a facial muscle, an eyebrow, a bone, a diaphragm, a finger, and a hand.

21. The device of claim 17, wherein the actuator comprises a waterproof outer cover.

22. The device of claim 17, wherein the electroactive polymer is selected form the group consisting of: an electro-statically stricted polymer, an electronic electroactive polymer, an ionic electroactive polymer, polyaniline, polypyrrole and polyacetylene.

23. The device of claim 17, wherein the implant comprises a sling, a cord, a membrane, a pulley or a lever.

24. The device of claim 17, wherein the biocompatible implant comprises a material selected from the group consisting of: a ligament, a connective tissue, a suture, polyvinyl chloride, silicone, polyurethane, polytetrafluoroethylene, polycarbonate, polyester, polyethylene, a hydrogel, titanium, and stainless steel.

25. The device of claim 17, further comprising a voltage source in electrical contact with the actuator, wherein the voltage source is selected from the group consisting of: a battery, a fuel cell, and an induction coil.

26. A method of using an electroactive prosthetic device comprising:
providing an electroactive polymer actuator functionally attached to an implant;
attaching the implant to one or more transposition points of an internal tissue of a living animal;
mounting the actuator to an internal anchoring location on the animal; and,
applying a voltage to the actuator thereby mechanically translating the implant and the tissue.

27. The method of claim 26, wherein the anchoring location is a bone of the animal.

28. The method of claim 27, wherein the actuator is mounted at an anchoring location above an eye orbit or lateral to an eye orbit.

29. The method of claim 26, wherein the one or more transposition points are selected from the group consisting of: skin connective tissue, a bone, a muscle attachment point, and a tendon.

30. The method of claim 26, wherein said applying of voltage is in response to a signal from a sensor.
